(19) United States
(12) Patent Application Publication
(10) Pub. No.: US 2015/0209317 A1
(43) Pub. Date: Jul. 30, 2015

(54) Plant Extracts for Improving Cognitive Function

(71) Applicant: Kemin Industries, Inc., Des Moines, IA (US)

(72) Inventors: Michael Ceddia, Ankeny, IA (US); Kelli Herrlinger, Johnston, IA (US)

(21) Appl. No.: 14/609,907

(22) Filed: Jan. 30, 2015

Related U.S. Application Data
(60) Provisional application No. 61/933,583, filed on Jan. 30, 2014.

(51) Int. Cl. A61K 31/216; A61K 36/534; A61K 36/53

(52) U.S. Cl. CPC A61K 31/216; A61K 36/53; A61K 36/534

(57) ABSTRACT

Extracts of plants of spearmint (Mentha spicata) are shown to improve memory, reasoning, attention/concentration and planning.

This application claims priority to U.S. Patent Application Ser. No. 61/933,583, filed Jan. 30, 2014, and incorporated herein in its entirety by this reference.

### BACKGROUND OF THE INVENTION

The invention relates generally to plant extracts that enhance, improve or sustain cognitive health and function and, more specifically, to the administration of an extract of spearmint (Mentha spicata L.) containing rosmarinic acid to improve memory, reasoning, attention/concentration, planning and associated behaviors.

There is a strong demand for products that can improve cognitive health or function and the market for these products has continued to grow in recent years despite the unfavorable economic pressures. Some of this growth can be attributed to growth of aging population, which is especially true in Asia and the US. Worldwide cognitive health ingredient sales are around $455 million. Frost and Sullivan have predicted an annual growth rate in this area to 12% from 2016 to 2019.

Major ingredients for cognitive health currently include phosphatidylserine (PS), CoQ10, omega-3 (marine oils/algae oils), citicoline, ginko and ginseng. Of the largest cognitive health ingredients, phosphatidylserine is the only one with a FDA approved qualified claim. With increasing scientific evidence to support the claim, the ingredient has be enjoying double digit growth in sales. In 2010, DHA and EPA health claims for brain function, heart health and vision obtained a positive opinion from EFSA in Europe. Citicoline is promoted as an ingredient that prevents neuronal degeneration and improves memory.

Rosmarinic acid (RA) is one of the major components found in spearmint and is an important contributor to its antioxidant capacity (Fletcher et al. Heat stress reduces the accumulation of rosmarinic acid and the total antioxidant capacity in spearmint (Mentha spicata L). Journal of the Science of Food and Agriculture 85: 2429-2436, 2005). RA, a naturally occurring phenolic compound, is an ester of caffeic acid and 3,4-dihydroxyphenyllactic acid. Its structure consists of a carbonyl group, unsaturated double bond, and carboxylic acid between two phenolic rings. RA has shown several biological activities, such as anti-inflammatory, anti-mutagenic, antibacterial, antidepressant, HIV-1 inhibitory, antioxidant, and antiviral properties. These properties have made RA an attractive ingredient for the pharmaceutical and cosmetic industries. RA has been used topically in Europe as a non-steroidal anti-inflammatory drug (Ritschel et al. Percutaneous absorption of rosmarinic acid in the rat. Methods and Findings in Experimental and Clinical Pharmacology 11: 345-352, 1989). Due to its extensive use as a flavoring agent and preservative in the food industry, RA is regarded as a daily-consumed safe ingredient (Alkam et al. A natural scavenger of peroxynitrites, rosmarinic acid, protects against impairment of memory induced by Aβ25-35. Behavioural Brain Research 180: 139-145, 2007).

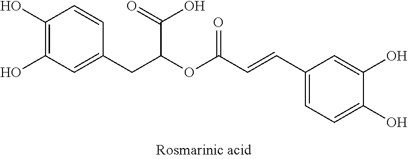 [SMILES: O=C(/C=C/c1ccc(O)c(O)c1)OC(Cc1ccc(O)c(O)c1)C(=O)O]

Evidence of RA's non-specific protective properties has been found within the brain. Improved anti-oxidant activity of the brain was demonstrated following RA administration to aging mice which resulted in increased activities of superoxide dismutase (SOD) and catalase (CAT) in the brain, while decreasing malondialdehyde (MDA) (Shou et al. Rosmarinic acid attenuates D-galactose induced behavior impairment in mice and its mechanism. 2010, p. 1723-1726). These data demonstrate the non-specific protective properties of RA as an antioxidant; however, no previous data has demonstrated RA's ability to affect the brain in specific regions or on specific clinical outcomes.

In vivo, three studies have evaluated administration of RA. These studies have administered RA either orally or IP in intracranial injury models or a stress model that were used to represent specific cognitive disease states (Alkam et al. A natural scavenger of peroxynitrites, rosmarinic acid, protects against impairment of memory induced by Aβ25-35. Behavioural Brain Research 180: 139-145, 2007; Park et al. Subchronic administration of rosmarinic acid, a natural prolyl oligopeptidase inhibitor, enhances cognitive performances. Fitoterapia 81:644-648, 2010; Zhou et al. Rosmarinic acid attenuates D-galactose induced behavior impairment in mice and its mechanism. Intl Conf BMEI 4:1723-1726, 2010). Although RA showed benefit in these models, they are not a validated model for evaluation of normal aging cognitive changes. In addition, it is unknown if the mechanisms of action are specific or non-specific due to antioxidant effects. Currently there are no published human studies evaluating RA supplementation alone or through use of a spearmint extract.

Learning and memory can be divided into two main categories, declarative and procedural. Declarative has temporal, spatial and associative memory components. This relates to learning and memory that has a conscious component requiring attention and alertness. In humans this relates to the acquisition, recognition and memory of discrete events, places, people, and facts. Procedural learning and memory can be formed when a declarative memory task becomes routine or habitual and was measured in the current animal study through the lever press. This relates to learning and memory that does not have a conscious component, which in humans is a habit or skill, such a riding a bike. Declarative tasks are thought of as hippocampal initiated, while procedural tasks are primarily linked to the caudate regions of the brain.

Memory impairment may occur in healthy, elderly individuals, and is considered a normal consequence of aging. In older subjects (>50 years), Gallo et al. reported that self-reported memory impairment occurs at a rate of approximately 20% (Gallo J J, Morales K H, Bogner H R, Raue P G, Zee J, Bruce M L, Reynolds C F. Long term effect of depression care management on mortality in older adults: follow-up of cluster randomized clinical trial in primary care. BMJ 2013; 346:f2570). Interestingly, in a cross-sectional study of 17 general practice clinics serving 2,934 patients aged 65 and older, 23% of these individuals self-reported memory impairment upon prompting; however, only 18% (of the 23%) had consulted a physician for their memory problem (Waldorff F B, Rishoi S, Waldemar G. If you don't ask (about memory), they probably won't tell. J Fam Pract 2008; 57(1):41-4.). Cognitive decline is generally accepted as a natural consequence of aging; however, it significantly decreases quality of life (Grossi D, Postiglione A, Schettini B, Trojano L, Barbarulo A M, Guigliano V, Ambron E, Aiello A. Autobiographical recall training in elderly adults with subjective memory complaint: a pilot study. Percept Mot Skills 2007; 104(2):621-8). It is estimated that 5.4 million elderly Americans have cognitive impairment without dementia and roughly 12% of these individuals will develop dementia annually (Plassman B L, Langa K M, Fisher G G, Heeringa S G, Weir D R, Ofstedal M B, Burke J R, Hurd M D, Potter G G, Rodgers W L, Steffens D C, Mcardle J J, Willis R J, Wallace R B. Prevalence of cognitive impairment without dementia in the United States. Ann Intern Med 2008; 148(6):427-34). Although a number of treatments are available for dementia, this prominent health concern emphasizes a need to explore methods to increase, maintain, or reduce the decline in cognitive function that is associated with aging.

Traditional medicine has long used plant-based remedies to treat a number of ailments and, more recently, plant-based remedies such as Ginko biloba (Wesnes K A, Ward T, Mcginty A, Petrini O. The memory enhancing effects of a Ginkgo biloba/Panax ginseng combination in healthy middle-aged volunteers. Psychopharmacology (Berl) 2000; 152(4):353-61; Snitz B E, O'meara E S, Carlson M C, Arnold A M, Ives D G, Rapp S R, Saxton J, Lopez O L, Dunn L O, Sink K M, Dekosky S T. Ginkgo biloba for preventing cognitive decline in older adults: a randomized trial. JAMA 2009; 302(24):2663-70), ginseng (Wesnes 2008; Reay J L, Kennedy D O, Scholey A B. Single doses of Panax ginseng (G115) reduce blood glucose levels and improve cognitive performance during sustained mental activity. J Psychopharmacol 2005; 19(4):357-65; Kennedy D O, Haskell C F, Robertson B, Reay J, Brewster-Maund C, Luedemann J, Maggini S, Ruf M, Zangara A, Scholey A B. Improved cognitive performance and mental fatigue following a multi-vitamin and mineral supplement with added guarana (Paullinia cupana). Appetite 2008; 50(2-3):506-13), and guarana (Kennedy 2004; Haskell 2007) have been investigated in clinical trials for their potential in enhancing cognitive function in healthy volunteers. A recent meta-analysis of 13 randomized controlled trials suggests herbal medicines provide a small but consistent benefit relative to placebo and are just as effective as pharmaceutical interventions in improving cognitive function in subjects with dementia (May B H, Lit M, Xue C C, Yang A W, Zhang A L, Owens M D, Head R, Cobiac L, Li C G, Hugel H, Story D F. Herbal medicine for dementia: a systematic review. Phytother Res 2009; 23(4):447-59). Furthermore, several trials have been conducted suggesting that consumption of plants within the Lamiaceae (mint) family, including lemon balm (Kennedy D O, Scholey A B, Tildesley N T, Perry E K, Wesnes K A. Modulation of mood and cognitive performance following acute administration of Melissa officinalis (lemon balm). Pharmacol Biochem Behav 2002; 72(4):953-64), lavender (Moss M, Cook J, Wesnes K, Duckett P. Aromas of rosemary and lavender essential oils differentially affect cognition and mood in healthy adults. Int J Neurosci 2003; 113(1):15-38), sage (Tildesley N T, Kennedy D O, Perry E K, Ballard C G, Savelev S, Wesnes K A, Scholey A B. Salvia lavandulaefolia (Spanish sage) enhances memory in healthy young volunteers. Pharmacol Biochem Behav 2003; 75(3):669-74; Tildesley N T, Kennedy D O, Perry E K, Ballard C G, Wesnes K A, Scholey A B. Positive modulation of mood and cognitive performance following administration of acute doses of Salvia lavandulaefolia essential oil to healthy young volunteers. Physiol Behav 2005; 83(5):699-709; Scholey A B, Tildesley N T, Ballard C G, Wesnes K A, Tasker A, Perry E K, Kennedy D O. An extract of Salvia (sage) with anticholinesterase properties improves memory and attention in healthy older volunteers. Psychopharmacology (Berl) 2008; 198(1):127-39), and rosemary (Pengelly A, Snow J, Mills S Y, Scholey A, Wesnes K, Butler L R. Short-term study on the effects of rosemary on cognitive function in an elderly population. J Med Food 2012; 15(1):10-7), may promote cognitive function in healthy volunteers. Compounds in the essential oils of plants within the Lamiaceae family, such as menthone, piperitone oxide, camphor, linalool, and polyphenols, are likely responsible for the wide range of reported biological activity of these plant extracts (Mimica-Dukic N, Bozin B, Sokovic M, Mihajlovic B, Matavulj M. Antimicrobial and antioxidant activities of three Mentha species essential oils. Planta Med 2003; 69(5):413-9; Hussain A I, Anwar F, Nigam P S, Ashraf M, Gilani A H. Seasonal variation in content, chemical composition and antimicrobial and cytotoxic activities of essential oils from four Mentha species. J Sci Food Agric 2010; 90(11):1827-36). However, randomized controlled trials specifically investigating the effects of spearmint on cognitive function are limited to a few studies which suggest spearmint-flavored chewing gum improves memory in healthy volunteers; albeit the evidence is conflicting (Baker J R, Bezance J B, Zellaby E, Aggleton J P. Chewing gum can produce context-dependent effects upon memory. Appetite 2004; 43(2):207-10; Tucha O, Mecklinger L, Maier K, Hammerl M, Lange K W. Chewing gum differentially affects aspects of attention in healthy subjects. Appetite 2004; 42(3):327-9; Miles C, Johnson A J. Chewing gum and context-dependent memory effects: a reexamination. Appetite 2007; 48(2):154-8; Johnson A J, Miles C. Chewing gum and context-dependent memory: the independent roles of chewing gum and mint flavour. Br J Psychol 2008; 99(Pt 2):293-306).

An efficacy study was conducted on the spearmint extract proposed in the current pilot trial to evaluate its potential in improving learning and memory in a SAMP8 mouse model of accelerated aging (U.S. patent application Ser. No. 13/962,609, filed Aug. 8, 2013, and incorporated herein by this reference). SAMP8 mice were administered spearmint extract or vehicle control. In addition, a 50% SAMP8 backcross strain served as the control which also received vehicle. After 90 days of treatment mice were tested in 3 behavioral tests which included, T-maze foot shock avoidance, object recognition and lever press. The spearmint extract improved acquisition (at both 16 and 32 mg of active/kg body weight) and retention (at all doses) in T-maze. In addition, spearmint with rosmarinic acid improved object recognition at 16 and 32 mg/kg body weight. The mouse doses of 16 and 32 mg of active/kg body weight correlate to a human equivalent dose of 91-180 mg rosmarinic acid, or 600-1200 mg of spearmint extract containing approximately 15% of the active. The results indicated that the extracts from spearmint have beneficial effects on deficits in learning and memory that occur with age in SAMP8 mice.

Safety studies were conducted on the spearmint extract to be utilized in the current human clinical pilot study (spearmint extract containing 15.4% [w/w] of rosmarinic acid) at Vanta Biosciences (Chennai, India) following the OECD and the FDA Redbook 2000 guidelines. The studies conducted included the Ames bacterial reverse mutation assay, chromosomal aberration induction assay, and 14-d and 90-d oral gavage toxicity study. Genotoxicity testing results demonstrate that spearmint extract was non-mutagenic at concentrations up to 5000 μg/plate as measured by the Ames bacterial reverse mutation assay. In addition, the spearmint extract did not demonstrate chromosomal aberration induction potential (non-clastogenic) at dose levels up to 5000 μg/ml.

Daily oral administration of the spearmint extract to male and female Sprague Dawley rats at doses up to 600 mg rosmarinic acid/kg body weight/day for 14 days was well tolerated. No test item-induced adverse effects were detected. The “No Observed Adverse Effect Level (NOAEL)” for the test item under the testing conditions was found to be 3896.1 mg/kg body weight/d of the spearmint extract. A follow-up 90-d study utilizing oral administration of the spearmint extract to male and female Sprague Dawley rats at doses up to 1948 mg rosmarinic acid/kg body weight/day for 90 d was well tolerated. The “No Observed Adverse Effect Level (NOAEL)” for the test item under the testing conditions and doses employed was found to be 300 mg rosmarinic acid/kg body weight/day (corresponding to 1948.2 mg/kg body weight/d of the spearmint extract). Utilizing a 100-fold safety factor this correlates to a human equivalent dose of 19.48 mg spearmint extract/kg body weight/day or a 1364 mg spearmint extract dose for a 70 kg human, which is greater than the dose proposed in the current study.

Currently, spearmint is widely used as an additive in beverages and confectioneries and has Generally Recognized as Safe status as a natural seasoning/flavoring, essential oil, or natural extract in the United States (FDA 2012a. Substances generally recognized as safe: Essential oils, oleoresins (solvent-free), and natural extractives (including distillates). 12CRF182.20; FDA 2012b. Substances generally recognized as safe: Spices and other natural seasonsings and flavorings. 12CRF182.10). However, the safety and tolerability of spearmint in humans when consumed at doses higher than what would typically be consumed as a flavoring or seasoning have not been evaluated. Thus the purpose of this pilot study is to evaluate the safety and tolerability of consumption of 900 mg of a spearmint extract, and the effects on cognitive function in healthy men and women with self-reported memory impairment.

### SUMMARY OF THE INVENTION

The invention consists of the administration of an extract of plants that contains rosmarinic acid to improve memory, reasoning, attention/concentration, planning and cognitive associated behaviors (i.e., focus, alertness, exploration, motivation, and the like). Spearmint extract with rosmarinic acid shows improved memory, reasoning, attention/concentration and planning in humans with self-reported memory impairment, both acutely and chronically.

Oxidative damage is considered one of the hallmarks of the aging process. The neuronal dysfunction present in cognitive impairments associated with aging is thought in large part to be from oxidative stress. Both structural and functional damage to mitochondria is present in cognitive disorders, such as Alzheimer's disease, suggesting that antioxidants that penetrate both the cell and the mitochondria will provide the greatest protection from oxidative stress. The current study was designed to test if a novel, proprietary antioxidant-based ingredient spearmint extract standardized to rosmarinic acid, could improve in humans with self-reported memory impairment.

A spearmint extract was standardized to contain 15% rosmarinic acid (minimum 13%). The extract was administered to provide 900 mg of the spearmint extract (135 mg rosmarinic acid) per day to each subject in the form of two 450 mg capsules. Spearmint extract as used in this application is available commercially from Kemin Industries, Inc. (Des Moines, Iowa) and is included in the commercial products FORTRA™ Dry and Neumentix™ Phenolic Complex K110-42. The subjects were evaluated on day 0 pre-administration and at 2 hours and 4 hours post administration (acute evaluation) and on day 30 pre-administration and at 2 hours and 4 hours post administration, the last day of the trial (chronic evaluation). In response to a subjective global improvement questionnaire, the subjects reported a subjective improvement after 30 days. There was no significant difference in mean individual or composite gastrointestinal tolerability scores over 30 days for any of the three possible groups analysis.

Subjects were evaluated using a variety of accepted tests of memory, reason, attention/concentration and planning. On an acute basis, the subject group, both modified intent to treat and per protocol, showed significant (p<0.1) improvement in reasoning, concentration and planning, and on a chronic evaluation basis both modified intent to treat and per protocol, showed significant (p<0.1) improvement in memory, reasoning, concentration and planning.

The current results indicate that the extract from spearmint (rosmarinic acid) has beneficial effects on memory, reasoning, attention/concentration and planning in humans with no adverse effects.

### BRIEF DESCRIPTION OF THE FIGURES

FIG. 1 presents a chart of the open-label clinical trial outline used to obtain the reported data.

FIG. 2 is a chart of cognitive function scores at baseline and at the end of a 30-day period of spearmint extract supplementation.

### DESCRIPTION OF THE INVENTION

The current study was designed to test if spearmint extract (with RA) could improve memory, reasoning, attention/concentration and planning in human subjects with self-reported memory impairment. Adverse events were monitored and a blood profile was taken at the end of the study.

This application incorporates by reference in their entirety U.S. Patent Application Ser. No. 62/050,523, filed Sep. 15, 2014, and Ser. No. 13/962,609, filed Aug. 13, 2013, Ser. No. 13/367,863, filed Feb. 7, 2012, and Ser. No. 13/367,888 filed Feb. 2, 2012.

### DEFINITIONS

As used in this application, the following terms have the meanings set out below.

Cognitive Health: Cognitive health refers to the health of the overall brain, tissues and blood supply as well as its' ability to function appropriately under various conditions. Good cognitive health is vital for the brain to perform all mental processes; collectively known as cognition including, but not limited to, learning, intuition, judgment, language, attention, alertness, focus and memory (both long and short-term); at peak performance. Poor cognitive health due to aging, diseases and/or other cognitive detriments reduce the brain's ability to function appropriately resulting in significant declines in cognitive function and performance.

Cognitive Function: Any mental or intellectual process involving neurological or symbolic operations including, but not limited, to communication, perception, comprehension, reasoning, memory, thinking, awareness, focus, attention, alertness, motivation, drawing conclusions, executive function, creation of imagery and capacity for judgment. In animal model systems, cognitive function may be measured in various conventional ways known in the art, including using a Morris Water Maze (MWM), Barnes circular maze, elevated radial arm maze, T-maze or any other mazes in which the animals use spatial information. Other tests known in the art may also be used to assess cognitive function, such as novel object recognition and odor recognition tasks.

Executive Function: Cognitive processes that regulate, control, and manage other cognitive processes, such as planning, working memory, attention, problem solving, verbal reasoning, mathematical ability, inhibition, mental flexibility, task switching, initiation, flexibility, visual attention, math skills, adaptability to new and changing environments and monitoring of actions.

Learning: The act, process, or experience of gaining knowledge or skill; psychological or behavioral modification especially through experience or conditioning.

Memory: The collection of information gained from past learning or experience that is stored in a person's mind. A piece of information, such as the mental image of an experience, that is stored in the memory. The ability to remember past experiences or learned information, involving advanced mental processes such as learning, retention, recall, and recognition and resulting from chemical changes between neurons in several different areas of the brain, including the hippocampus. Included are (1) declarative learning or memory, which refers to which can be consciously recalled such as facts and knowledge, (2) working memory, which refers to actively holding multiple pieces of transitory information in the mind where they can be manipulated, (3) reference memory, which refers to information gained from previous experience, either recent or remote, (4) recognition memory, which is the ability to recognize previously encountered events, objects, or people, and (5) associative memory, which is the ability to learn and remember the relationship between unrelated items. Each of these has and immediate, short-term, and long-term component. Immediate memory lasts for just a few seconds. Short-term memory stores information that has been minimally processed and is available only for a few minutes, as in remembering a phone number just long enough to use it. Short-term memory is transferred into long-term memory, which can last for many years, only when repeated use of the information facilitates neurochemical changes that allow it to be retained.

Therapeutically effective amount: The amount of a compound or composition or derivatives thereof of the present invention is an amount that, when administered to a subject, will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of the cognitive impairment, and the therapeutics or combination of therapeutics selected for administration, and the mode of administration. The skilled worker can readily determine the effective amount for a given situation by routine experimentation. In one embodiment, the at least one extract of a plant of the Lamiaceae family as described herein are for administration, for example, RA, on a daily frequency or more than once a day, e.g. 2, 3 or 4 times a day

Treatment or Treating: Clinical intervention in an attempt to alter the natural course of the individual, animal or cell being treated, and may be performed either for prophylaxis or during the course of clinical pathology. Desirable effects include preventing occurrence or recurrence of disease, alleviation of symptoms, or diminishment of any direct or indirect pathological consequences of the disease, lowering the rate of disease progression, amelioration or palliation of the disease state, and remission or improved prognosis. A condition or subject refers to taking steps to obtain beneficial or desired results, including clinical results. Beneficial or desired clinical results include, but are not limited to, enhancing, improving or sustaining cognitive health and/or function, alleviation or amelioration of one or more symptoms associated with mild cognitive impairment, or age-related cognitive impairment, delay or slowing of that impairment, amelioration, palliation or stabilization of that impairment, and other beneficial results, such as improvement of cognitive function or a reduced rate of decline of cognitive function in subjects with age-related cognitive impairment or at risk thereof. In preferred embodiments, these terms include the prevention or treatment of cognitive disorders such as dyslexia, aspraxia, attention-deficit-hyperactivity disorder, attention-deficit disorder autism, Alzheimer's, Parkinson's or stroke, or other disorders of executive function.

In preferred embodiments of the present invention, the dosage of rosmarinic acid ranges from 9 mg/day to 7,000 mg/day and all values between such limits, including, for example, without limitation or exception, 10, 10.4, 13.2, 21.7, 33.6, 48.9, 137.7, 433.2, 913.2, 1,254.6, 3,555.3, 5,021.3 and 6,990.9 mg/day Stated another way, in preferred embodiments of the invention, the dosage can take any value “abcd” mg/day wherein a is selected from the numerals 0, 1, 2, 3, 4, 5, 6 and 7, and b, c and d are each individually selected from the numerals 0, 1, 2, 3, 4, 5, 6, 7, 8 and 9, with the exception that d cannot be less than 9 if a, b, and c are all 0.

Where ranges are used in this disclosure, the end points only of the ranges are stated so as to avoid having to set out at length and describe each and every value included in the range. Any appropriate intermediate value and range between the recited endpoints can be selected. By way of example, if a range of between 0.1 and 1.0 is recited, all intermediate values (e.g., 0.2, 0.3. 6.3, 0.815 and so forth) are included as are all intermediate ranges (e.g., 0.2-0.5, 0.54-0.913, and so forth).

### Example 1

Oxidative damage is considered one of the hallmarks of the aging process [Harman D (2002) Alzheimer's disease: role of aging in pathogenesis. Ann N Y Acad Sci. 959, 384-395]. The neuronal dysfunction present in diseases associated with aging such as Alzheimer's disease is thought in large part to be from oxidative stress [Markesbery W R (1997) Oxidative stress hypothesis in Alzheimer's disease. Free Radic Biol Med. 23, 134-147; Polidori M C, Griffiths H R, Mariani E, Mecocci P (2007) Hallmarks of protein oxidative damage in neurodegenerative diseases: focus on Alzheimer's disease. Amino Acids. 32, 553-559]. Both structural and functional damage to mitochondria is present in Alzheimer's disease suggesting that antioxidants that easily penetrate both the cell and the mitochondria will provide the greatest protection from oxidative stress [Skulachev V P, Anisimov V N, Antonenko Y N, Bakeeva L E, Chernyak B V, Erichev V P, Filendo O F, Kalinia N I, Kapelko V I, Kolosova N G, Kopin B P, Korshunova G A, Lichinitser M R, Obukhova L A, Pasyukova E G, Pisarenko O I, Roginsky V A, Ruuge E K, Senin I I, Severina I I, Skulachev M V, Spivak I M, Tashlitsky V N, Tkachuk V A, Vyssokikh M Y, Yaguzhinsky L S, Zorov D B (2009) An attempt to prevent senescence: a mitochondrial approach. Biochim Biophys Acta. 1787, 437-461; Suh J H, Shigeno E T, Morrow J D, Cox B, Rocha A E, Frei B, Hagen T M (2001) Oxidative stress in the aging rat heart is reversed by dietary supplementation with (R)-(alpha)-lipoic acid. FASEB J. 15, 700-706].

Rosmarinic acid (RA) has been found to be neuroprotective and preventative against oxidative stress [Fadel O, El Kirat K, Morandat S (2011) The natural antioxidant rosmarinic acid spontaneously penetrates membranes to inhibit lipid peroxidantion in situ. Biochim Biophys Acta 1808, 2973-2980; Fallarini S, Miglio G, Paoletti T, Minassi A, Amoruso A, Bardelli C, Brunelleschi S, Lombardi G (2009) Clovamide and rosmarinic acid induce neuroprotective effects in invitro models of neuronal death. Br J Pharmacol 157, 1072-1084. Protection against oxidative stress and inflammation has been associated with improved memory in diseases of aging [Farr, et al., 2012]. Rosmarinic acid improved memory in the Morris water maze spatial task [Park D H, Park S J, Kim J M, Jung W Y, Ryu J H (2010) Subchronic administration of rosmarinic acid, a natural prolyl oligopeptidase inhibitor, enhances cognitive performance. Fitoterapia 81, 644-648].

Memory is divided in to two main categories declarative (or explicit memory) and procedural (or implicit memory). Declarative memory is further subdivided into semantic (facts or meaning) and episodic (specific experiences). Semantic memory is generally derived from episodic memory. Declarative memories are thought of as being encoded by the hippocampus whereas procedural memories are thought of as being encoded by the caudate a structure within the striatum. Procedural or implicit memory comes from learning the association between a response and a reward. Procedural memories often start as declarative memories until they become ingrained or a habit.

### Materials and Methods

### Subjects.

At the start of treatment, the subjects for the experiments were 11, 50-70 year old men and women with self-reported memory impairment. Eligible participants were 73% female and 27% male with a mean age and body mass index (BMI) of 58.7 y and 27.4 kg/m2, respectively. These studies were conducted at Biofortis Clinical Research, Addison, Ill.

### Study Design.

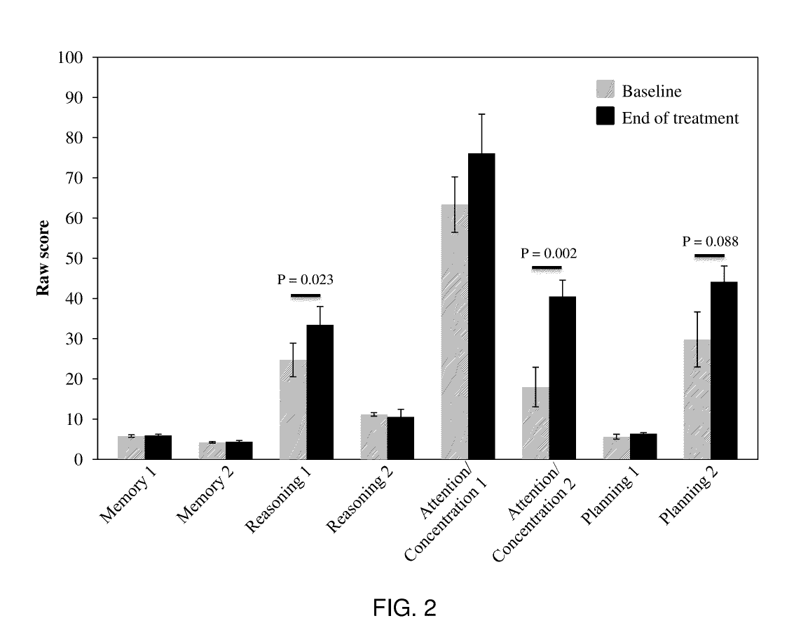

The open-label study included one telephone screen (TS; Appendix 1—the reference to appendices is to those that were set out in U.S. Pat. Appl. Ser. No. 61/933,583, the disclosure of which is incorporated herein in its entirety); one screening visit (visits 1a/b; day −7); one baseline visit (visit 2; day 0); and one test visit (visit 3; day 30). There was a ±3 d window for all clinic visits. At the TS (Appendix 1), the paper Memory Complaint Questionnaire (MAC-Q; Crook 1992; Appendix 3) was administered to assess for self-reported memory impairment. Eligible subjects (MAC-Q score ≧25; Dunbar 2007) came to the clinic (visit 1a, day −7) fasting (10-14 h prior to the start of blood draw, water only), provided informed consent and were administered the paper Mini Mental State Examination (MMSE; Folstein 1975, Mitrushina 1991). Eligible subjects (scoring ≧24 on the MMSE; Dunbar 2007) continued on with visit 1b (day −7), by undergoing the remaining screening visit procedures including, evaluations of medical history, inclusion and exclusion criteria, prior and current medication/supplement use, height, body weight, and vital signs. If a subject normally takes antihypertensive medication(s), the medication should be taken at the clinic prior to blood being drawn. Vital signs were assessed at least 30 min after the administration of the antihypertensive medication(s). Fasting (10-14 h, water only) blood samples were collected in the morning for a chemistry profile, hematology panel, and lipid profile with samples stored as a backup for possible future analysis of non-genetic indicators of metabolism. Female subjects <60 years of age underwent an in-clinic urine pregnancy test. Subjects performed at least two practice computerized cognitive test batteries, ˜1 h per session each separated by ≧1 h for training purposes. A maximum of four practice test batteries were allowed to ensure that the subject is familiar with the testing procedure and to ensure an optimal level of performance prior to the first computerized cognitive test (Owen 2010; Appendices 5 and 6; FIGS. 2-4 of U.S. Pat. Appl. Ser. No. 61/933,583) at the baseline visit (visit 2, day 0; Appendix 5). Written study instructions were provided [fasting compliance (1014 h, water only); avoidance of vigorous physical activity (24 h), consumption of alcoholic beverages (24 h), caffeine intake (10-14 h) and tobacco use (1 h) prior to, and for the duration of each test visit (visits 2 and 3; days 0 and 30), and maintenance of habitual diet (including consumption of caffeine and alcohol), physical activity patterns, sleep duration, and sleep aid medication/supplement intake]. Additionally, if the subject normally takes a sleep aid medication/supplement, subjects were advised to keep the intake of the sleep aid medication/supplement consistent the night before each test visit (visits 2 and 3; days 0 and 30). Subjects were asked to schedule the visit 2 (day 0) clinic visit prior to departing the clinic.

Eleven eligible subjects were enrolled to the 900 mg spearmint extract group followed by the administration of a paper Gastrointestinal (GI) Tolerability Questionnaire (Appendix 8) and the first computerized cognitive tests at t=−1.0 h±5 min, where t=0 h is the time of study product ingestion. At t=0 h, subjects were administered the study product immediately followed by a standard breakfast meal. Subjects consumed the meal in its entirety within 15 min. The standard breakfast meal foods/amounts were replicated (i.e., exact foods/amounts from the visit 2, day 0 breakfast will be served) at visit 3 (day 30). Subjects were provided with a standard amount of water following the completion of the standard breakfast meal. Subjects were allowed to drink water ad libitum throughout the test visit, except for when actually at the computer undergoing the cognitive testing. Actual water consumption was recorded.

### Outcome Variables Primary Outcome Variables.

The co-primary outcome variables are to estimate the change from baseline (visit 2, day 0) to end of treatment (visit 3, day 30) in the gastrointestinal (GI) tolerability composite score derived from the GI Tolerability Questionnaire (nausea, gas/bloating, flatulence, GI cramping, constipation, and diarrhea/loose stools) and to evaluate the overall SGI composite score (a subjective cognitive assessment) obtained at the end of treatment (visit 3, day 30).

### Secondary Outcome Variables.

Secondary outcome variables include: (a) Changes from baseline to the end of the treatment (day 30) for individual scores of the GI Tolerability Questionnaire (nausea, gas/bloating, flatulence, GI cramping, and diarrhea/loose stools); and (b) acute (differences from t=−1 to t=2.25 and 4.0 h) and (c) chronic (differences from visit 2, day 0 to visit 3, day 30) changes in cognitive function individual test scores [raw and performance (percentile) ratings] using a publicly available computerized cognitive assessment tool designed and validated at the Medical Research Council Cognition and Brain Sciences Unit (Owen 2010; cambridgebrainsciences.com), including Memory: Digit Span and Paired Associates, Reasoning: Double Trouble and Odd One Out, Attention/Concentration (Rotations and Polygons) and Planning (Spatial Search and Spatial Slider). Individual SGI scores for memory, attention, and speed of thinking were taken at the end of treatment (visit 3, day 30).

### Safety and Tolerability Measurements.

Safety and tolerability will be assessed by evaluation of treatment-emergent adverse events (AEs), body weight, vital signs, and changes in clinical laboratory measurements.

### Analyses.

A statistical analysis plan was generated and approved prior to database lock. All statistical analyses were conducted using SAS for Windows (Cary, N.C.). The safety population included all subjects who were enrolled into the study and consumed at least one dose of study product. The modified intent-to-treat (MITT) population comprised all subjects included in the safety population who provided at least one on-treatment outcome data point during the treatment phase. In addition, the per protocol (PP) population was comprised of a subset of the MITT population. Subjects were excluded from the PP population for the following reasons: Violations of inclusion or exclusion criteria that could influence the evaluation of response; and non-compliance by the subject, including, but not limited to missing appointments, less than 80% or greater than 120% compliance with study product consumption, failure to consume the study product in its entirety at any test visit (visits 2 and 3; days 0 and 30), and use of prohibited drugs or any products thought to alter the outcome variables during the study.

All tests of significance, unless otherwise stated, were performed at alpha <0.1, two-sided. The paired t-test or Wilcoxon signed rank test was used, as appropriate to test whether or not the changes are statistically significant. The objectives were to gain information on the means/medians and variability in the study endpoints.

Safety was assessed by AEs reported by subjects at all on-treatment clinic visits, as well as changes in vital signs measurements, laboratory values, and body weight. AEs were coded by the World Health Organization (WHO) dictionary. Missing data was imputed and only observed data was included in the statistical analysis.

### Sample Size.

A sample of 11 subjects were enrolled into the study.

### Study Design and Procedures.

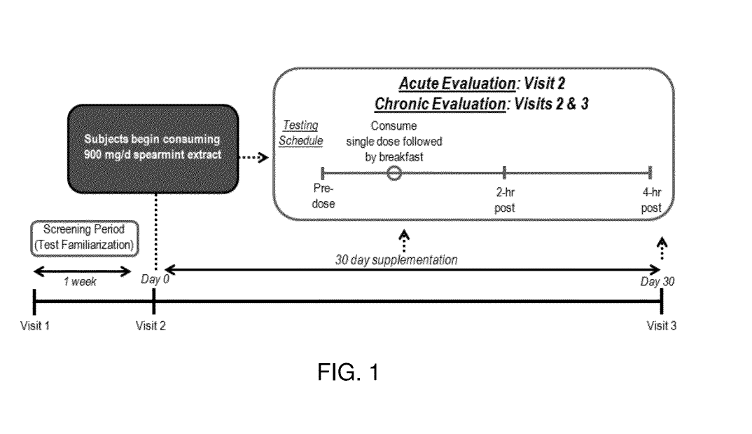

A schematic diagram of the study design is illustrated in FIG. 1.

This open-label study included one telephone screen (TS; Appendix 1); one screening visit (visits 1a/b; day −7); one baseline visit (visit 2; day 0); and one test visit (visit 3; day 30). There were a ±3 d window for all clinic visits. At the TS, the paper Memory Complaint Questionnaire (MAC-Q; Crook 1992; Appendix 3) was administered to assess for self-reported memory impairment. Eligible subjects (MAC-Q score ≧25; Dunbar 2007) came to the clinic (visit 1a, day −7) fasting (10-14 h prior to the start of blood draw, water only), provide informed consent and be administered the paper Mini Mental State Examination (MMSE; Folstein 1975, Mitrushina 1991). Eligible subjects (scoring ≧24 on the MMSE; Dunbar 2007) continued on with visit 1b (day −7), by undergoing the remaining screening visit procedures including, evaluations of medical history, inclusion and exclusion criteria, prior and current medication/supplement use, height, body weight, and vital signs. If a subject normally takes antihypertensive medication(s), the medication should be taken at the clinic prior to blood being drawn. Vital signs were assessed at least 30 min after the administration of the antihypertensive medication(s). Fasting (10-14 h, water only) blood samples were collected in the morning for a chemistry profile, hematology panel, and lipid profile with samples stored as a backup for possible future analysis of non-genetic indicators of metabolism. Female subjects <60 years of age underwent an in-clinic urine pregnancy test. Subjects performed at least two practice computerized cognitive test batteries, ˜1 h per session each separated by ≧1 h for training purposes. A maximum of four practice test batteries were allowed to ensure that the subject was familiar with the testing procedure and to ensure an optimal level of performance prior to the first computerized cognitive test (Owen 2010) at the baseline visit (visit 2, day 0; Appendix 5). Written study instructions were provided [fasting compliance (10-14 h, water only); avoidance of vigorous physical activity (24 h), consumption of alcoholic beverages (24 h), caffeine intake (10-14 h) and tobacco use (1 h) prior to, and for the duration of each test visit (visits 2 and 3; days 0 and 30), and maintenance of habitual diet (including consumption of caffeine and alcohol), physical activity patterns, sleep duration, and sleep aid medication/supplement intake]. If there was greater than a ±2 h deviation from the subject's average sleep duration at night (as reported at the screening visit), the test visit may have been rescheduled. Additionally, if the subject normally takes a sleep aid medication/supplement, subjects were advised to keep the intake of the sleep aid medication/supplement consistent the night before each test visit (visits 2 and 3; days 0 and 30). Subjects were asked to schedule the visit 2 (day 0) clinic visit prior to departing the clinic.

At visit 2 (day 0±3 d), eligible subjects arrived at the clinic fasted (10-14 h, water only, anchored to t=−1.25 h timepoint between 0600-0930 h. Following clinic visit procedures (i.e., assessment of inclusion and exclusion criteria, concomitant medication/supplement use, body weight, and vital signs), subjects were queried on compliance with the aforementioned study instructions. Adverse events (AEs) were assessed and sleep duration was queried to assess if there is greater than a ±2 h deviation from the subject's average sleep duration at night, reported at the screening visit (visit 1b; day −7). If subjects were not 100% compliant with the study instructions the day prior to visit 2 (day 0), subjects were counseled on the need for appropriate compliance for the remaining test visits and the test visit may have been rescheduled. If a subject normally takes antihypertensive medication(s) in the morning, the medication was taken at the clinic 30 min prior to the vital sign measurements. Eligible subjects were enrolled to the 900 mg spearmint extract group followed by the administration of the paper Gastrointestinal (GI) Tolerability Questionnaire (Appendix 8) and the first computerized cognitive test (Appendix 5) at t=−1.0 h±5 min, where t=0 h is the time of study product ingestion. At t=0 h, subjects were administered the study product immediately followed by a standard breakfast meal (Appendix 7). Subjects will consume the meal in its entirety within 15 min. The standard breakfast meal foods/amounts were replicated (i.e., exact foods/amounts from the visit 2, day 0 breakfast were served) at visit 3 (day 30). Subjects were provided with a standard amount of water following the completion of the standard breakfast meal. Subjects were allowed to drink water ad libitum throughout the test visit, except for when actually at the computer undergoing the cognitive testing. Actual water consumption was recorded.

At t=2.25 and 4.0 h±5 min, where t=0 h is the time of study product ingestion, the computerized cognitive test was administered (Owen 2010; Appendices 5 and 6). Following the t=4 h±5 min cognitive function tests, AEs were assessed and written study instructions were provided [fasting compliance (10-14 h, water only); avoidance of vigorous physical activity (24 h), consumption of alcoholic beverages (24 h), caffeine intake (10-14 h) and tobacco use (1 h) prior to, and for the duration of subsequent test visit (visit 3; day 30), and maintenance of habitual diet (including consumption of caffeine and alcohol), physical activity patterns, sleep duration and sleep aid medication/supplement intake]. If there was greater than a ±2 h deviation from the subject's average sleep duration at night, the test visit was rescheduled. Additionally, if the subject consumed a sleep aid medication/supplement the night prior to visit 2 (day 0), the subject were advised to keep this intake consistent the night before visit 3 (day 30).

Subjects were dispensed the study product bottle (from which the morning dose was administered) with instructions to consume two capsules/d with breakfast until the next visit (visit 3, day 30). Subjects were also instructed to record study product consumption and sleep duration in a daily Study Diary (Appendix 11) and were provided with a pill box to aid with daily compliance. Additionally, subjects were reminded that they would be contacted weekly by telephone throughout the trial to ensure compliance with the study product, study instructions, and to assess any AEs and/or changes in daily habits (i.e., medication/supplement(s), eating, sleeping, and/or exercise). If a study product dose was missed within three days of the subsequent test visit, the test visit was rescheduled.

At visit 3 (day 30±3 d), subjects arrived at the clinic between 0600-0930 h. Fasting (10-14 h, water only) blood samples were collected at t=−1.25 h±5 min for assessment of the chemistry profile, hematology panel and a lipid profile. If a subject normally takes medication in the morning, the medication was taken at the clinic 30 min prior to the vital sign measurements. Clinic visit procedures were conducted (i.e., assessment of inclusion and exclusion criteria, concomitant medication/supplement use, body weight, and vital signs) and subjects were queried on compliance with study instructions and AEs were assessed. Study product and the Study Diary (Appendix 11) were collected and compliance were determined. The paper GI Tolerability Questionnaire, paper Subject Global Improvement Questionnaire (SGI; Dunbar 2011, Lieberman 2013; Appendices 8 and 9), and the computerized cognitive tests (Owen 2011; Appendices 5 and 6) were administered at t=−1.0 h±5 min, where t=0 h is the time of study product ingestion. At t=0 h, subjects were administered their assigned study product immediately followed by a standard breakfast meal (foods/amounts from visit 2, day 0 breakfast were served). Subjects consumed the meal in its entirety, including the study product, within 15 min. The study product and standard breakfast meal at visit 3 (day 30) were administered within ±30 min of the t=0 h time established at visit 2 (day 0). were At t=2.25 and 4.0 h±5 min, where t=0 h is the time of study product ingestion, the computerized cognitive test were administered (Owen 2010; Appendices 5 and 6). Subjects were provided with a standard amount of water following the completion of the standard breakfast meal. Subjects were allowed to drink water ad libitum throughout the test visit, except for when actually at the computer undergoing the cognitive testing. Actual water consumption were recorded. Following the t=4.0 h±5 min cognitive test, AEs were assessed.

### Study Sample.

Each subject was required to meet all of the following inclusion criteria and none of the exclusion criteria at baseline in order to participate in this study.

### Inclusion Criteria

1 Subject is male or female, 50-70 years of age, inclusive.

2 Subject meets the criteria for self-reported memory impairment based on the MAC-Q (score ≧25; Crook 1992).

3 Subject has a body mass index (BMI) 18.5-35.0 kg/m, inclusive, at visit 1b (day −7).

4 Subject has at least a high school diploma or the equivalent.

5 Subject is willing to maintain a habitual diet (including caffeine and alcohol) and physical activity patterns throughout the study period, except for the 24 h prior to each test day (visits 2 and 3; days 0 and 30).

6 Subject has no health conditions that would prevent him/her from fulfilling the study requirements as judged by the Investigator on the basis of medical history and routine laboratory test results.

7 Subject is willing to comfortably abstain from tobacco products for at least 1 h prior to and throughout the duration of the test visits [visits 2 and 3 (days 0 and 30) for up to 7 h].

8 Subject is willing to eat breakfast at test visits (visits 2 and 3; days 0 and 30) and at home on a daily basis throughout the study period.

9 Subject is willing and able to comfortably abstain from caffeine prior to (1014 h) and throughout the duration of all clinic visits (visits 1b, 2 and 3; days 7, 0, and 30).

10 Subject is willing to abstain from alcohol consumption and avoid vigorous physical activity for 24 h prior to all clinic visits (visits 1b, 2, and 3; days −7, 0 and 30).

11 Subject is judged by the Investigator to be in general good health on the basis of medical history.

12 Subject understands the study procedures and signs forms providing informed consent to participate in the study and authorization for release of relevant protected health information to the study Investigators.

Exclusion Criteria

1 Subject has an abnormal laboratory test result of clinical significance, including, but not limited to creatinine ≧1.5 mg/dL and ALT or AST≧1.5× upper level of normal at visit 1b (day −7). Clinically relevant laboratory test results will be treated as an AE, upon the Investigators discretion. Subjects will be advised to follow up with their Primary Care Physician.

2 Subject is unable to understand and/or completely perform the practice tests.

3 Subject scores ≦23 on the MMSE at visit 1b (day −7; Folstein 1995; Mitrushina 1991, Dunbar 2007).

4 Subject experiences evidence of delirium, confusion, or other disturbances of consciousness.

5 Subject has a history of diagnosed depression in the prior 2 years of visit 1b (day −7).

6 Subject has any neurologic disorder that could produce cognitive deterioration including, but not limited to, Alzheimer's disease, Parkinson's disease, stroke, intracranial hemorrhage, local brain lesions including tumors, and normal pressure hydrocephalus.

7 Subject has a history of any infective or inflammatory brain disease, including those of viral, fungal, or syphilitic etiologies.

8 Subject has a history of repeated minor head injury (e.g., in boxing) or a single injury resulting in a period of unconsciousness for 1 h or more.

9 Subject has elective hospitalizations planned (e.g., elective cosmetic procedures) during the study period.

10 Subject has uncontrolled hypertension (systolic blood pressure ≧160 mm Hg or diastolic blood pressure ≧100 mm Hg as defined by the average blood pressure measured at visit 1b (day −7). One re-test will be allowed on a separate day prior to visit 2 (day 0), for subjects whose blood pressure exceeds either of these cut points at visit 1b, in the judgment of the Investigator.

11 Subject has a history or presence of a clinically relevant cardiac, renal, hepatic, endocrine (including diabetes mellitus), pulmonary, biliary, gastrointestinal, pancreatic, or neurologic disorder.

12 Subject has a history or presence of cancer in the prior 2 years, except for non-melanoma skin cancer.

13 Subject has an active infection or signs/symptoms of an infection at any clinic visit. Clinic visits will be rescheduled to allow subject to be symptom-free of any type of systemic infection for at least 5 d.

14 Subject has recently used antibiotics (within 5 d of any clinic visit).

15 Subject is a heavy smoker, defined as a history of smoking >1 pack-per-day in the 3 months prior to visit 1b (day −7).

16 Subject is a heavy consumer of caffeinated beverages (>400 mg caffeine/d from caffeine-containing products) within 2 weeks of visit 1b (day −7).

17 Subject has inconsistently used sleep aid products the day prior to each test visit (visits 2 and 3; days 0 and 30). Test visits will be rescheduled should this occur.

18 Subject has a sleep disorder (e.g., sleep apnea) or occupation where sleep during the overnight hours is irregular (e.g., 3^{rd} shift of overnight workers).

19 Subject has a deviation of ±2 h from their normal sleep duration the evening prior to test visits 2 and 3 (days 0 and 30). Test visits will be rescheduled should this occur (personal communication: Patrick O'Connor, University of Georgia, and Kevin Maki, Biofortis).

20 Subject has a known allergy or sensitivity to the study product or any ingredients of the standard meal provided.

21 Subject has a history of use of psychotropic medications (including antidepressants and tranquilizers) within 4 weeks of visit 1b (day −7) and throughout study period.

22 Use of antioxidants or other supplements with the potential to influence cognitive function within 2 weeks of visit 1b (day −7) and throughout the study.

23 Subject has a recent history of (within 12 months of visit 1b, day −7) or strong potential for alcohol or substance abuse. Alcohol abuse defined as >14 drinks per week (1 drink=12 oz beer, 5 oz wine, or 11/2 oz distilled spirits).

24 Subject is a female who is pregnant, planning to be pregnant during the study period, lactating, or is of childbearing potential and is unwilling to commit to the use of a medically approved form of contraception throughout the study period. The method of contraception must be recorded in the source documentation.

25 Subject has had exposure to a non-registered drug product within 30 d prior or 5 half-lives of elimination, whichever is longer, to the screening visit (visit 1a, day −7).

26 Individual has a condition the Investigator believes would interfere with his or her ability to provide informed consent, comply with the study protocol, which might confound the interpretation of the study results, or put the subject at undue risk.

Excluded Medications and Products

Psychotropic medications were not allowed within four weeks of visit 1b (day −7) (Appendix 2). Additionally, use of antioxidants or other supplements with the potential to influence cognitive function were excluded within 2 weeks of visit 1b (day −7) and throughout the study period. Antibiotic therapy had to be completed at least five days prior to any clinic visit (visits 1b, 2 and 3; days −7, 0, and 30). Subjects were also be instructed to avoid caffeine and caffeine-containing products for 10-14 h prior to any clinic visit (visits 1a/1b, 2, and 3; days −7, 0, 30) and for the duration of each clinic visit. Additionally, subjects were advised to avoid alcohol (24 h), vigorous physical activity (24 h), and tobacco use (1 h) prior to, and for the duration of any test visit (visits 2 and 3; days 0 and 30).

Medications taken on an “as needed” basis were not to be taken on the morning of any clinic visit (visits 1b through 3; days −7 through 30). Any necessary medication that subjects normally take in the morning were taken at the clinic in the presence of study staff, at the Investigator's discretion, and with the following guidelines for antihypertensive medications:

Visit 1b (day −7): If a subject normally takes antihypertensive medication, the medication was taken at the clinic prior to blood being drawn for the chemistry profile, hematology panel, and lipid profile. Vital signs were assessed at least 30 min after the administration of the medication.

Visits 2 and 3 (days 0 and 30): If a subject normally takes medication(s) in the morning, the medication was taken at the clinic 30 min prior to the vital sign measurement. If the subject's blood pressure was elevated when vitals were assessed, blood pressure assessment was repeated approximately 30 min after the time the antihypertensive medication was administered. Timing of medication was monitored.

### Study Product.

Description

Nine hundred milligrams per day (900 mg/d) proprietary spearmint extract (2-450 mg capsules) standardized to contain 67.5 mg rosmarinic acid per capsule.

The first and last administration of the study product was performed in-clinic at visits 2 and 3 (days 0 and 30) with a standard breakfast. The remaining study product (two capsules) were self-administered at home. Study product information for the study product can be found in Appendix 10.

Storage and Dispensing/Use of Study Product

Study products were stored in a locked, dry secure location (59-86° F.). Study product supplies were to be used only in accordance with this protocol and under the supervision of the Investigator. At the conclusion of the study, all unused study product were returned to the sponsor.

Subjects will receive 80 capsules dispensed at visit 2 (day 0). This will provide subjects with enough study product to allow for flexibility in scheduling the next clinic visit. Subjects were required to return all unused study product at visit 3 (day 30).

### Clinical Measurements.

Laboratory Measurements

The procedures for all clinical laboratory measurements were described in detail in a laboratory manual developed for Elmhurst Memorial Hospital Laboratory (Elmhurst, Ill.) and other laboratory vendors. Normal reference ranges were provided in the laboratory instructions.

The following were performed as a part of the fasting (10-14 h) chemistry panel: glucose, sodium, potassium, chloride, CO2, BUN, creatinine, calcium, osmolality, AST, ALT, alkaline phosphatase, total bilirubin, total protein, albumin (Appendix 4). Samples were stored frozen for backup and for possible later analysis of non-genetic markers.

The following were performed as part of the fasting (10-14 h) hematology measurements: WBC, WBC with differential, RBC, hemoglobin, hematocrit and platelet count.

Fasting (10-14 h) lipids (TC, LDL-C, HDL-C, non-HDL-C, and TG) were analyzed according to the Standardization Program of the Centers for Disease Control and Prevention and the National Heart, Lung and Blood Institute. LDL-C and the concentration in mg/dL were calculated according to the Friedewald equation (Friedewald 1972) as follows:

LDL-C=TC-HDL-C-TG/5

Since this equation is not valid when the TG concentration is above 400 mg/dL, no LDL-C value was calculated under these circumstances. Samples were stored frozen for backup and for possible later analysis of non-genetic markers.

Clinically relevant laboratory test results were treated as an AE, upon the Investigators discretion.

Clinic Visits

Clinic visit assessments included height (visit 1b only), body weight, concomitant medication/supplement use, and where appropriate, and evaluations of inclusion/exclusion criteria. There was a ±3 d window for clinic visits.

Standardized vital signs measurements included resting blood pressure and pulse measured using an automated blood pressure measurement device using the same arm for each measurement. Blood pressure was obtained after the subject has been sitting for at least five min. Subjects were required to refrain from smoking cigarettes during the 60 min preceding the measurement. Systolic and diastolic pressures were measured twice using an appropriate sized cuff (bladder within the cuff must encircle ≧80% of the arm), separated by at least one min. Both measurements were recorded. If elevated blood pressure occurred at the screening visit (visit 1b, day −7), one retest was allowed on a separate day. Heart rate was measured twice using an automated blood pressure measurement device.

Screening Memory Questionnaires

During the telephone screen (within 2 weeks of visit 1a, day −7) subjects were administered the paper MAC-Q, a 6-point question designed to assess self-reported memory impairment (score ≦24 is exclusionary; Appendix 3; Crook 1992, Dunbar 2007). Eligible subjects continued with visit 1a (day −7) by providing informed consent, followed by administration of the paper MMSE (visit 1a; day −7; Folstein 1975, Mitrushina 1991).

The MMSE is a brief 30-point questionnaire test that is used to screen for cognitive impairment. The questionnaire will screen to exclude individuals with dementia (exclusionary score ≦23; Dunbar 2007). The questionnaire takes approximately 10 min to administer and measures cognitive function with arithmetic, memory and orientation domains.

Study Instructions/Query

Written study instructions [fasting compliance (10-14 h, water only); avoidance of vigorous physical activity (24 h), consumption of alcoholic beverages (24 h), caffeine intake (10-14 h) and tobacco use (1 h) prior to, and for the duration of each clinic visit (visits 1a, 2, and 3; days −7, 0, and 30), and maintenance of habitual diet (including consumption of caffeine and alcohol), physical activity patterns, sleep duration and sleep aid medication/supplement intake] were provided at the end of each clinic visit in preparation for the subsequent clinic visit. If there is greater than a ±2 h deviation from the subject's average sleep duration at night (as reported at the screening visit), the test visit was rescheduled. Additionally, if the subject consumed a sleep aid medication/supplement the night prior to visit 2 (day 0), the subject was advised to keep this intake consistent the night before visit 3 (day 30). Study instructions were reviewed during the weekly telephone contact calls.

Adverse Event Assessment

AE assessment occurred at the beginning of the baseline and final test visits (visits 2 and 3; days 0 and 30), and at the conclusion of these visits following the completion of the final cognitive testing at t=4.0 h. AEs were assessed during the weekly telephone contact. Inquiring about AEs occurred with an open-ended question. At the beginning of visits 2 and 3 (days 0 and 30), study staff inquired about any major change/life stress event. In the case of a major life change/stress (e.g., death of a family member) the visit may be rescheduled if, in the opinion of the Investigator, cognition could be affected.

Cognitive Testing Familiarization

The practice test took ˜1 h per session, each administration separated by ≧1 h. The practice test was for training purposes, in order to familiarize each subject with the testing procedure.

Cognitive Testing

The Cambridge Brain Sciences computerized tests are publicly available cognitive assessment tools validated at the Medical Research Council and Brain Sciences Unit (Cambridge, UK; Owen 2010). Cognitive testing included the assessment of Memory (Digit Span and Paired Associates); Reasoning (Double Trouble and Odd One Out); Attention/Concentration (Rotations and Polygons); and Planning (Spatial Search and Spatial Slider; Appendices 5 and 6) at t=−1.0, 2.25, and 4.0 h±5 min, for a total of three cognitive test batteries, where t=0 h is the time of study product ingestion. Subjects were tested in the same room at each test administration and test visit. Environmental conditions such as lighting, heating, and noise were kept as constant as possible during testing and across test visits.

Study Product/Standard Breakfast Administration

At test days (visits 2 and 3; days 0 and 30), eligible subjects arrived at the clinic fasted (10-14 h) between 0600-0930 h. Following clinic visit procedures and cognitive functioning testing/questionnaires, subjects were administered their assigned study product at t=0 h, immediately followed by a standard breakfast meal. Subjects consumed the meal in its entirety, including the study product within 15 min. The study product and standard breakfast meal at visit 3 (day 30) were administered within ±30 min of the t=0 h time established at visit 2 (day 0) and the menu were replicated (i.e., exact foods/amounts from the visit 3, day 0 breakfast were served). Subjects were provided with a standard amount of water following the standard breakfast meal. Subjects were allowed to drink water ad libitum throughout the test visit, except for when actually at the computer undergoing the cognitive testing. Actual water consumption was recorded.

Study Diary

Subjects completed a daily Study Diary to keep track of sleep duration and study product intake (following visit 2; day 0 until the end of the study; Appendix 11).

Gastrointestinal Tolerability Questionnaire

A paper GI Tolerability Questionnaire was administered prior to the standard breakfast meal/study product consumption at visits 2 and 3 (days 0 and 30; Maki 2008). Subjects will respond to questions to assess the presence and severity of selected GI symptoms, including gas/bloating, nausea, flatulence, diarrhea/loose stools, constipation, and GI cramping.

Subject Global Improvement Questionnaire

Subjects were administered the paper SGI Questionnaire (Dunbar 2011, Lieberman 2013) at visit 3 (day 30; Appendix 9) at t=−1.0 h±5 min, where t=0 h is the time of study product ingestion. Subjects were required to answer questions regarding global improvement relating to memory, attention, and speed of thinking.

Dispense Study Product

Subjects were dispensed the study product (from which the morning dose was administered) with instructions to consume two capsules with breakfast every day throughout the 30 d treatment period. Subjects were dispensed a daily Study Diary at visit 2 (day 0; Appendix 11). Subjects were instructed to record study product intake. A pill box was supplied to each subject to aid with daily compliance.

Telephone Contact

Subjects were contacted on a weekly basis throughout the trial to ensure compliance with the study product, study instructions, and to assess any AEs and/or changes in daily habits (i.e., medications/supplements, eating, sleeping, and/or exercise). Documentation of the telephone contact was recorded in the subject source document and case report form (CRF).

Test Day Schedule

| Test time | |
|---|---|
| (t = “x” h) | BASELINE/TEST DAYS (visits 2 and 3; days 0 and 30) |
| 6:30 h | • Arrive at clinic; |
| 9:30 h | • Study instructions query; • Clinic visit procedures; • AE assessment; • Enrollment |
| | (visit 2, day 0 only); • Review Study Diary (visit 3; day 30 only); • Collect Study |
| | Product/Assess Compliance (visit 3; day 30 only) |
| t = −1.25 h ± 5 min | • Chemistry profile (visit 3, day 30 only); |
| | • Hematology panel (visit 3, day 30 only); • Lipid profile (visit 3, day 30 only) |
| t = −1.0 h + 5 min | • Paper GI Tolerability Questionnaire; • Paper SGI Questionnaire (visit 3, day 30 |
| | only) • Computerized Cognitive Testing; |
| t = 0 h | • Single-dose study product administered immediately followed by a standard |
| | breakfast |
| t = 2.25 and 4.0 h ± 5 min | • Computerized Cognitive Testing; |
| | • Ad libitum water consumption allowed following the completion of the standard |
| | breakfast meal. Subjects will be allowed to drink water ad libitum throughout the |
| | test visit, except for when actually at the computer undergoing the cognitive testing. |
| | Actual water consumption will be recorded. |
| Following t = 4.0 h timepoint | • AE assessment; |
| | • Dispense study product (visit 2; day 0 only); • Dispense Study Diary (visit 2; day 0 |
| | only); • Review study instructions (visit 2; day 0 only) |

Data Analysis and Statistical Methods.

Outcome Analysis

All tests of significance, unless otherwise stated, were performed at alpha <0.1, two-sided.

Descriptive statistics were presented for values at all timepoints and all changes (within days and across days). The paired t-test or Wilcoxon signed rank test were used, as appropriate to test whether or not the changes were statistically significant. However, this is a pilot study and it is understood that the power is inadequate for statistical testing. The objectives were to gain information on the means/medians and variability in the study endpoints.

Safety and Tolerability Analysis

Safety and tolerability were assessed by AEs reported by subjects at all on-treatment clinic visits, as well as changes in vital signs measurements, laboratory values, and body weight. AEs were coded by the World Health Organization (WHO) dictionary. Missing data will not be imputed and only observed data were included in the statistical analysis.

Results

In total, 20 participants were screened for this trial and 11 subjects met the inclusion and none of the exclusion criteria. Of the 11 subjects who were enrolled in the study, one subject withdrew consent after the baseline test visit due to inability to understand the cognitive function tests and was removed from the PP sample. A second subject was removed from the PP sample due to 134% compliance for study product consumption. A single adverse event, back pain, was reported during the treatment period and coded as unrelated to the study product consumption.

Baseline characteristics of the MITT sample (N=11) and the subset of subjects (n=5) included in the exploratory outcome analyses are included in Table 2.

TABLE 2
Baseline characteristics of subjects
in the overall and subset samples

| | | |
|---|---|---|
| | Overall Value | Subset |
| Parameter | (N = 11) | (n = 5) |

| | n (%) | | | |
|---|---|---|---|---|
| Male | 3 | (27) | 3 | (60) |
| Female | 8 | (73) | 2 | (40) |
| Race/Ethnicity | | | | |
| Non-Hispanic White | 10 | (91) | 5 | (100) |
| Black/African American | 1 | (9) | 0 | (0) |
| | Mean (SEM) | | | |
| Age (years) | 58.7 | (1.6) | 56.0 | (2.4) |
| Body mass index (kg/m²) | 27.4 | (1.0) | 25.6 | (0.8) |
| MAC-Q score | 29.7 | (1.0) | 29.0 | (1.3) |
| MMSE score | 28.9 | (0.4) | 28.4 | (0.5) |

The sample was comprised of 27% males and 73% females, with mean age and BMI of 58.7±1.6 y and 27.4±1.0 kg/m², respectively. Mean overall compliance with study product consumption was 103.2±1.6% and 98.3±1.0% for the MITT and subset samples, respectively. Mean scores for the qualifying MAC-Q and MMSE were 29.7±1.0% and 28.9±0.4% in the MITT sample, respectively.

Mean scores from the GI Tolerability Questionnaire which assessed changes in presence and severity of GI symptoms over the 30 d treatment period are shown in Table 3.

TABLE 3
Gastrointestinal Tolerability Questionnaire scores
at baseline, end of treatment, and change from baseline
in response to spearmint supplementation¹

| | Baseline² | EOT³ | | |
|---|---|---|---|---|
| | (N = 11) | (n = 10) | Difference (Δ) | P- |
| Parameter | Median (Interquartile Limits) | | | value4 |
| Constipation | 0.0 (0.0, 0.0) | 0.0 (0.0, 0.0) | 0.0 (0.0, 0.0) | 1.000 |
| Cramping | 0.0 (0.0, 0.0) | 0.0 (0.0, 0.0) | 0.0 (0.0, 0.0) | 1.000 |
| Flatulence | 0.0 (0.0, 0.0) | 0.0 (0.0, 0.0) | 0.0 (0.0, 0.0) | 1.000 |
| Gas/Bloating | 0.0 (0.0, 0.0) | 0.0 (0.0, 0.0) | 0.0 (0.0, 0.0) | 1.000 |
| Loose stools | 0.0 (0.0, 0.0) | 0.0 (0.0, 0.0) | 0.0 (0.0, 0.0) | 1.000 |
| Nausea | 0.0 (0.0, 0.0) | 0.0 (0.0, 0.0) | 0.0 (0.0, 0.0) | 1.000 |
| Composite score5 | 0.0 (0.0, 0.0) | 0.0 (0.0, 0.0) | 0.0 (0.0, 0.0) | 1.000 |

Abbreviations: EOT, end of treatment; SEM, standard error of the mean.
¹The GI Tolerability Questionnaire composite score was calculated for each test condition as the sum of the ratings for each parameter and coded as: −2 = less than usual; −1 = somewhat less than usual; 0 = none experienced/usual, 1 = somewhat more than usual, 2 = much more than usual.
²Baseline refers to pre-dose values on day 0.
³End of treatment (EOT) refers to pre-dose values on day 30.
4P-values were calculated from paired t-tests or Wilcoxon sign rank test, between baseline and end of treatment.
5The composites score is the sum of all ratings greater than or equal to 0.

Consumption of the spearmint extract did not significantly alter individual GI symptoms (constipation, cramping, flatulence, gas/bloating, loose stools, and nausea) between baseline and the end of treatment (P=1.000 for all comparisons). In addition, the GI tolerability composite score did not change significantly between baseline and the end of treatment (P=1.000). Mean scores from the SGI Questionnaire which assessed change from baseline in three domains of cognition (memory, attention, and speed of thinking) are shown in Table 4.

TABLE 4
Subject Global Impression Scale of Cognition Questionnaire scores
at the end of treatment in response to spearmint supplementation¹

| Parameter | Mean (SEM) | P-value² |
|---|---|---|
| Memory | 3.7 (0.2) | 0.500 |
| Attention | 3.5 (0.2) | 0.125 |
| Speed of thinking | 3.4 (0.3) | 0.125 |
| Average score | 3.5 (0.2) | 0.063 |

Abbreviations: SEM, standard error of the mean.
¹The Subject Global Impression (SGI) Scale of Cognition Questionnaire was administered at the end of the 30 d treatment and subjects were asked to compare their current condition to their condition prior to inclusion in the study. Scores were coded as: 1 = very much improved, 2 = much improved, 3 = minimally improved, 4 = no change, 5 = minimally worse, 6 = much worse, 7 = very much worse.
²P-values were calculated from Wilcoxon sign rank test, testing the difference from 4 (no change; n = 10) at the end of treatment.

The average composite score from the SGI Questionnaire improved slightly (3.5±0.3, a score of 4 represents ‘no change’; P=0.063) after 30 d of spearmint extract treatment. The difference in the average composite score (from a score of 4), was no longer significant in the PP sample (P=0.125). There were no significant differences in individual ratings from the SGI Questionnaire.

Mean and median values for vital signs, fasting lipoprotein lipids at baseline and end of treatment, and the change from baseline are presented in Table 5.

TABLE 5
Vital signs and fasting lipoprotein lipids at baseline, end of treatment,
and change from baseline in response to spearmint supplementation

| | Baseline¹ | | EOT² | | Difference | | |
|---|---|---|---|---|---|---|---|
| | (N = 11) | | (n = 10) | | (Δ) | | P- |
| Parameter | Mean (SEM) or Median (Interquartile Limits) | | | | | | value³ |
| SBP (mm Hg) | 121.1 | (3.6) | 121.7 | (3.3) | −0.9 | (2.4) | 0.706 |
| DBP (mm Hg) | 75.3 | (2.5) | 78.3 | (2.6) | 1.3 | (2.3) | 0.603 |
| Heart rate (bpm) | 63.2 | (2.2) | 68.0 | (2.7) | 3.7 | (1.8) | 0.077 |
| Body weight (kg) | 77.1 | (2.6) | 77.8 | (2.9) | 0.4 | (0.3) | 0.212 |
| LDL-C (mg/dL) | 138.6 | (11.5) | 148.6 | (11.4) | 5.2 | (5.4) | 0.361 |
| Non-HDL-C (mg/dL) | 156.6 | (10.8) | 163.7 | (11.6) | 3.1 | (5.5) | 0.584 |
| TC (mg/dL) | 213.1 | (11.5) | 222.3 | (12.5) | 3.4 | (6.0) | 0.586 |
| HDL-C (mg/dL) | 56.6 | (3.4) | 58.6 | (3.2) | 0.3 | (1.6) | 0.858 |
| Triglycerides (mg/dL) | 88.2 | (8.8) | 75.7 | (6.1) | −9.3 | (8.3) | 0.293 |
| TC/HDL-C | 3.7 | (3.1, 4.3) | 3.6 | (3.4, 4.5) | 0.1 | (−0.1, 0.3) | 0.432 |

Abbreviations: bpm, beats per minute; DBP, diastolic blood pressure; HDL-C, high-density lipoprotein cholesterol; LDL, low-density lipoprotein cholesterol; SBP, systolic blood pressure; TC, total cholesterol.
¹Baseline refers to pre-dose values on day 0.
²End of treatment (EOT) refers to pre-dose values on day 30.
³P-values were calculated from paired t-tests or Wilcoxon sign rank test, between baseline and end of treatment.

No significant differences in lipid parameters were evident over the 30 d treatment period in the MITT sample. An increase in LDL cholesterol in the PP sample was evident over the treatment period (8.9±4.4 mg/dL, baseline=137.0 mg/dL; P=0.079). Heart rate increased slightly over the 30 d treatment period (3.65±1.8 bpm; P=0.077); however, this change was no longer significant in the PP sample (P=0.155). Body weight increased over the 30 d treatment period in the PP sample only by 0.6±0.3 kg (P=0.062; baseline=77.0±3.2 kg). Blood chemistry and hematology values at baseline and end of treatment, and the change from baseline are presented in Tables 6 and 7.

TABLE 6
Absolute chemistry panel values at baseline, end of treatment, and
changes from baseline, in response to spearmint supplementation

| | Baseline¹ | | EOT² | | Difference (Δ) | | |
|---|---|---|---|---|---|---|---|
| Parameter | Mean (SEM) | | | | | | P-value³ |
| Glucose (mg/dL) | 94.9 | (1.5) | 97.3 | (2.1) | 1.6 | (1.5) | 0.318 |
| Sodium (mmol/L) | 140.3 | (0.4) | 140.2 | (0.3) | −0.1 | (0.5) | 0.847 |
| Potassium (mmol/L) | 4.4 | (0.1) | 4.6 | (0.1) | 0.1 | (0.2) | 0.447 |
| Chloride (mmol/L) | 104.9 | (0.6) | 105.5 | (0.8) | 0.2 | (0.9) | 0.836 |
| Carbon dioxide | 29.5 | (0.4) | 29.8 | (0.6) | 0.6 | (0.4) | 0.193 |
| (mmol/L) | | | | | | | |
| BUN (mg/dL) | 13.6 | (0.8) | 14.1 | (1.0) | 0.4 | (0.8) | 0.637 |
| Creatinine (mg/dL) | 0.9 | (0.0) | 0.9 | (0.1) | 0.0 | (0.0) | 0.515 |
| BUN/Creatinine | 15.7 | (1.2) | 16.4 | (1.8) | 0.5 | (1.6) | 0.785 |
| Anion gap (mmol/L) | 5.9 | (0.5) | 4.9 | (0.4) | −0.9 | (0.4) | 0.068 |
| Calcium (mg/dL) | 9.6 | (0.1) | 9.4 | (0.1) | −0.2 | (0.0) | 0.007 |
| Calcium osmolality | 290.6 | (0.8) | 290.7 | (0.8) | −0.1 | (1.1) | 0.931 |
| (mOs/kg) | | | | | | | |
| AST (U/L) | 22.2 | (1.5) | 22.6 | (1.8) | 0.4 | (1.4) | 0.786 |
| ALT (U/L) | 20.3 | (1.7) | 19.8 | (2.0) | −0.6 | (2.1) | 0.776 |
| ALP (U/L) | 69.9 | (5.7) | 66.7 | (5.6) | −1.5 | (1.0) | 0.169 |
| Total bilirubin (mg/dL) | 0.8 | (0.1) | 0.7 | (0.1) | −0.0 | (0.1) | 0.790 |
| Total protein (g/dL) | 6.9 | (0.2) | 6.7 | (0.2) | −0.2 | (0.1) | 0.055 |
| Albumin (g/dL) | 4.0 | (0.1) | 3.9 | (0.1) | −0.1 | (0.1) | 0.111 |
| Globulin (g/dL) | 2.9 | (0.2) | 2.9 | (0.2) | −0.1 | (0.1) | 0.421 |

Abbreviations: ALP, alkaline phosphatase; ALT, alanine transaminase; AST, aspartate aminotransferase; BUN, blood urea nitrogen; EOT, end of treatment.
¹Baseline refers to pre-dose values on day 0 (N = 11).
²End of treatment (EOT) refers to pre-dose values on day 30 (n = 10).
3P-values were calculated from paired t-tests or Wilcoxon sign rank test, between baseline and end of treatment.

TABLE 7
Absolute hematology panel values at baseline, end of treatment, and
changes from baseline, in response to spearmint supplementation

| | Baseline¹ | | EOT² | | Difference (Δ) | | |
|---|---|---|---|---|---|---|---|
| Parameter | Mean (SEM) or Median (Interquartile limits) | | | | | | P-value3 |
| WBC (cells/μL) | 5.6 | (4.1, 6.9) | 4.6 | (4.2, 6.3) | 0.3 | (−0.1, 0.4) | 0.447 |
| RBC (cells × 10⁶/μL) | 4.5 | (0.1) | 4.6 | (0.1) | 0.1 | (0.1) | 0.529 |
| Hemoglobin (g/dL) | 13.4 | (0.2) | 13.6 | (0.3) | 0.2 | (0.3) | 0.529 |
| Hematocrit (%) | 39.9 | (0.7) | 40.4 | (0.9) | 0.4 | (0.9) | 0.634 |
| MCV (fL) | 88.4 | (1.6) | 88.5 | (1.7) | −0.0 | (0.4) | 0.980 |
| MCH (pg/cell) | 29.7 | (0.7) | 29.8 | (0.7) | 0.1 | (0.2) | 0.648 |
| Platelets (cells × 10³/μL) | 221.7 | (8.6) | 222.5 | (11.2) | 0.3 | (2.5) | 0.906 |
| Neutrophils | 3.1 | (0.3) | 3.0 | (0.3) | −0.0 | (0.2) | 0.916 |
| (cells × 10³/μL) | | | | | | | |
| Lymphocytes | 1.7 | (0.1) | 1.7 | (0.2) | 0.0 | (0.1) | 0.713 |
| (cells × 10³/μL) | | | | | | | |
| Monocytes | 0.5 | (0.0) | 0.5 | (0.1) | 0.0 | (0.0) | 0.193 |
| (cells × 10³/μL) | | | | | | | |
| Eosinophils | 0.1 | (0.1, 0.2) | 0.1 | (0.1, 0.2) | 0.0 | (0.0, 0.0) | 0.500 |
| (cells × 10³/μL) | | | | | | | |
| Basophils (cells × 10³/μL) | 0.1 | (0.0, 0.1) | 0.1 | (0.0, 0.1) | 0.0 | (0.0, 0.0) | 1.000 |

Abbreviations: EOT, end of treatment; MCH, mean corpuscular hemoglobin; MCV, mean corpuscular volume; RBC, red blood cells; WBC, white blood cells.
¹Baseline refers to pre-dose values on day 0 (N = 11).
²End of treatment (EOT) refers to pre-dose values on day 30 (n = 10).
³P-values were calculated from paired t-tests or Wilcoxon sign rank test, between baseline and end of treatment.

Values from the blood chemistry panel revealed declines in calcium (−0.15±0.04 mg/dL, P=0.068), the anion gap (−0.9±0.4 mmol/L; P=0.007), and total protein (−0.2±0.1 g/dL; P=0.055) between baseline and end of treatment. No significant differences were evident over the 30 d treatment for whole blood hematology panel values.

Mean scores from the cognitive function tasks at baseline and end of treatment are represented in FIG. 2. Scores from reasoning 1, attention/concentration 2, and planning 2 cognitive function tasks improved significantly between baseline and the end of treatment by 6.4±4.2 (P=0.023), 22.9±5.3 (P=0.001), and 11.3±5.9 (P=0.088) points at the pre-dose assessment (t=−1 h), respectively. The change in the planning 2 task score was no longer significant in the PP sample (P=0.169). All other scores from the cognitive function tasks did not differ significantly between baseline and end of treatment.

Mean and median scores from the cognitive function tasks after acute and acute-on-chronic administration tests administered during the baseline and end of treatment test visits are represented in Table 8.

TABLE 8
Cognitive function scores after acute spearmint administration test at baseline and an acute-
on-chronic administration test at the end of a 30-d period of spearmint supplementation

| | Pre-dose | | 2.25 h post- | | 4 h post- | | P-value1 | P-value2 |
|---|---|---|---|---|---|---|---|---|
| Cognitive | score | | dose (Δ) | | dose (Δ) | | (pre-dose vs. | (pre-dose vs. |
| Test | Mean (SE) or Median (Interquartile limits) | | | | | | 2.25 h) | 4 h) |
| Baseline | | | | | | | | |
| (day 0) | | | | | | | | |
| Memory 1 | 5.8 | (0.3) | −0.2 | (0.2) | 0.1 | (0.3) | 0.441 | 1.000 |
| Memory 2 | 4.3 | (0.2) | 0.0 | (0.3) | −0.1 | (0.3) | 1.000 | 0.724 |
| Reasoning 1 | 24.7 | (4.2) | 2.6 | (1.5) | 3.1 | (2.0) | 0.129 | 0.161 |
| Reasoning 2 | 11.2 | (0.4) | −0.6 | (0.8) | −0.3 | (0.8) | 0.528 | 0.747 |
| Attention/ | 63.4 | (6.7) | 19.0 | (8.2) | 29.1 | (6.6) | 0.042 | 0.001 |
| Concentr. 1 | | | | | | | | |
| Attention/ | 18.0 | (4.9) | 16.8 | (6.4) | 21.8 | (5.3) | 0.025 | 0.002 |
| Concentr. 2 | | | | | | | | |
| Planning 1 | 6.0 | (5.0, 7.0) | 0.0 | (−1.0, 1.0) | 0.0 | (−1.0, 2.0) | 0.859 | 0.460 |
| Planning 2 | 29.8 | (6.9) | 0.4 | (5.5) | 11.7 | (3.2) | 0.949 | 0.004 |
| EOT | | | | | | | | |
| (day 30) | | | | | | | | |
| Memory 1 | 5.9 | (0.4) | 0.2 | (0.4) | 0.3 | (0.3) | 0.662 | 0.279 |
| Memory 2 | 4.4 | (0.3) | 0.2 | (0.3) | 0.0 | (0.3) | 0.555 | 1.000 |
| Reasoning 1 | 33.4 | (4.6) | 1.9 | (1.6) | 4.4 | (2.1) | 0.272 | 0.070 |
| Reasoning 2 | 12.5 | (11.0, 13.0) | −2.0 | (−4.0, 0.0) | −1.0 | (−3.0, 1.0) | 0.176 | 0.723 |
| Attention/ | 76.1 | (9.8) | 22.9 | (15.2) | 12.2 | (13.4) | 0.167 | 0.388 |
| Concentr. 1 | | | | | | | | |
| Attention/ | 40.5 | (4.1) | 1.1 | (4.5) | −10.0 | (5.6) | 0.813 | 0.108 |
| Concentr. 2 | | | | | | | | |
| Planning 1 | 6.4 | (0.3) | 0.5 | (0.4) | 0.2 | (0.4) | 0.213 | 0.642 |
| Planning 2 | 44.1 | (4.0) | −10.7 | (6.2) | 1.5 | (5.2) | 0.121 | 0.780 |

Abbreviations: EOT, end of treatment.
1P-values were calculated from paired t-tests or Wilcoxon sign rank test, between pre-dose (t = −1 h) and 2.25 h post-dose raw scores.
2P-values were calculated from paired t-tests or Wilcoxon sign rank test, between pre-dose (t = −1 h) and 4 h post-dose raw scores

The mean scores for attention/concentration 1 task increased between the pre-dose and post-dose timepoints (2.25 and 4 h) by 19.0±8.2 points (P=0.042) and 29.1±6.6 points (P=0.001), respectively, at the baseline test visit. Similarly, mean scores from the attention/concentration 2 task increased between the pre-dose and post-dose assessments (2.25 and 4 h) by 16.8±6.4 points (P=0.025) and 21.8±5.3 points (P=0.002), respectively, at the baseline test visit. Mean scores from the planning 2 task were also significantly elevated at the 4 h post-dose timepoint by 11.7±3.2 (P=0.004), relative to the pre-dose assessment at the baseline test visit. Mean scores from the reasoning 1 task increased between the pre-dose and post-dose assessments (2.25 and 4 h) in the PP sample only, by 3.4±1.7 points (P=0.082) and 4.9±2.0 points (P=0.041), respectively, at the baseline test visit. These acute improvements in cognitive function were not evident following the acute-on chronic administration test. However, an acute-on-chronic effect was evident in the reasoning 1 task at the 4 h post-dose assessment, scores improved by 4.40±2.1 points (P=0.070) relative to the pre-dose assessment. This difference was no longer significant in the PP sample (P=0.115).

### Discussion

In this open-label, pilot trial, consumption of spearmint extract daily 900 mg/d for 30 d was well-tolerated. Although significant differences were evident in LDL cholesterol, anion gap, calcium, total protein, heart rate, and body weight, these ranges are within normal biological variability and are likely not clinically meaningful. Modest improvements in subjective cognition were evident after 30 d of supplementation. The results of this trial suggest spearmint extract supplementation may improve aspects of cognitive function including reasoning, attention/concentration, and planning with chronic supplementation, as well as attention/concentration and planning acutely.

To the best of our knowledge, no previously published studies have evaluated the safety and tolerance of spearmint extract in humans at dose levels that exceed what would typically be consumed as an additive, seasoning, or flavoring. However, a few studies have evaluated spearmint toxicity in animal models. Specifically, in a study by Akodogan et al. rats (n=12/group) consumed spearmint tea (20 and 40 g/L) ad libitum or the vehicle water for 30 d (Akdogan M, Kilinc I, Oncu M, Karaoz E and Delibas N. Investigation of biochemical and histopathological effects of Mentha piperita L. and Mentha spicata L. on kidney tissue in rats. Human and Experimental Toxicology. 2003; 22:213-219). Plasma concentrations of urea and creatinine were significantly elevated (P<0.003) at both dose levels, relative to the control, following spearmint tea consumption. Similarly, a second study utilizing the same study design in rats also reported significant elevations in activity of hepatic enzymes at both dose levels, aspartate aminotransferase (AST) and alanine aminotransferase (ALT), relative to control (P<0.016) (Akdogan M, Ozguner M, Aydin G and Gokalp O. Investigation of biochemical and histopathological effects of Mentha piperita Labiatae and Mentha spicata Labiatae on liver tissue in rats. Human and Experimental Toxicology. 2004; 23:21-28). Spearmint intake was estimated at 2.2 g/kg body weight (20 g/L) and 4.4 g/kg body weight (40 g/L) per day in these studies, which roughly translates to a 25-50 g/d dose in 70 kg human (Reagan-Shaw S, Nihal M and Ahmad N. Dose translation from animal to human studies revisited. FASEB J. 2008; 22:659-661). Although, the estimated level of spearmint consumption in these animal studies is 3-fold higher than what was consumed in the current study, these findings were not confirmed.

Conflicting and limited evidence exists regarding spearmint and its effect on aspects of cognitive function. In a randomized controlled trial, healthy young participants (mean age=24.6 y; n=25/group) were assigned to one of three treatment groups: chewing of sugar-free spearmint gum, mimic chewing of gum, or avoid chewing (control) (Wilkinson L, Scholey A and Wesnes K. Chewing gum selectively improves aspects of memory in healthy volunteers. Appetite. 2002; 38:235-236). These conditions were carried out during administration of a computerized test battery of cognitive function tasks. The results showed that chewing spearmint gum improved subjects' memory relative to the control group (P<0.05) but did not suggest any differences in attention/concentration. In a second study, healthy young participants (mean age=22.9 y; n=20-23/group) were randomly assigned to one of four treatment conditions: chewing spearmint gum chewing flavorless gum, mimic chewing of gum, or no chewing (control) (Tucha O, Mecklinger L, Maier K, Hammerl M and Lange K W. Chewing gum differentially affects aspects of attention in healthy subjects. Appetite. 2004; 42:327-329). These conditions were also carried out during administration of a computerized test battery of cognitive function tasks. Contrary to the results of the first study, chewing spearmint gum did not improve memory but did improve sustained attention/concentration, relative to control (P<0.01). It is uncertain from these studies if the improvement in memory and attention/concentration is a result of the spearmint or the act of chewing. Further, the interpretation of these results is difficult given the lack of consistent dose levels, time of dosing, population, and the cognitive function assessment tools utilized. Specific acute improvements in memory tasks were not identified in the current study but acute improvements were evident with spearmint supplementation in attention/concentration tasks.

In conclusion, spearmint extract was well-tolerated in older subjects (50-70 y) with self-reported memory impairment and may positively impact cognitive function both acutely and chronically.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

We claim:

1. A method to enhance, improve or sustain cognitive health and/or function in mammals, comprising oral administration of an efficacious amount of an extract of a plant of the Lamiaceae family.

2. A method of claim 1, wherein said mammals are selected from the list consisting of humans and companion animals.

3. A method of claim 2, wherein said efficacious amount of said extract is between 0.1 and 100 mg/kg/day.

4. A method of claim 1, wherein said extract comprises rosmarinic acid.

5. A method of claim 4, wherein cognitive health and/or cognitive function refers to memory, reasoning, attention/concentration and planning.

6. A method of claim 1, wherein said plant is selected from the group consisting of basil, mint, sage, savory, marjoram, oregano, thyme, lavender, spearmint, and rosemary.

7. A method of claim 4 for prevention or treatment of cognitive disorders such as dyslexia, aspraxia, attention-deficit-hyperactivity disorder, attention-deficit disorder autism, Alzheimers, Parkinsons or stroke, or other disorders of executive function.

8. A method of claim 4 for prevention or treatment of cognitive disorders or deficits associated with the normal aging process.

9. A method of claim 4 for maintenance of normal cognitive function under a stressful condition such as disruption of circadian rhythms as occurs in a condition selected from the list consisting of jet lag, time zone changes, pregnancy, medications, changes in routine and shift work.

10. A method of claim 7 for maintenance of normal cognitive function under stressful condition such as disruption of circadian rhythms as occurs in a condition selected from the list consisting of time zone changes, pregnancy, medications, changes in routine and shift work.

11. A method to treat or prevent a decline in cognitive health and/or function in mammals, comprising oral administration of an efficacious amount of an extract of a plant of the Lamiaceae family.

12. A method of claim 1, wherein said extract is to be administered to a human subject and the human subject shows improvement in cognitive function as measured by an evaluation consisting of a computerized cognitive assessment tool for assessing attention/concentration via Rotations and/or Polygons.

13. A method of claim 1, wherein said extract is to be administered to a human subject daily over a period of at least 30 days and the human subject shows improvement in cognitive function as measured by at least one evaluation selected from the group consisting of subjective responses to a cognitive questionnaire, a computerized cognitive assessment tool for assessing memory via Digit Span and/or Paired Associates, a computerized cognitive assessment tool for assessing reasoning via Double Trouble and/or Odd One Out, a computerized cognitive assessment tool for assessing attention/concentration via Rotations and/or Polygons and a computerized cognitive assessment tool for assessing planning via Spatial Search and/or Spatial Slider.

14. A method of claim 1, wherein said plant is spearmint (Mentha spicata) comprising greater than 8 wt % rosmarinic acid on a dry weight basis.
